(19) United States
(12) Patent Application Publication
(10) Pub. No.: US 2020/0188280 A1
(43) Pub. Date: Jun. 18, 2020

(54) COMPOSITION AND METHOD FOR TEMPORARILY RESHAPING KERATINOUS FIBRES

(71) Applicant: Henkel AG & Co. KGaA, Duesseldorf (DE)

(72) Inventors: JULIA BIBIANE LANGE, Bad Bramstedt (DE); CYRIELLE MARTINEZ, Hamburg (DE); DIANE METTEN, Hamburg (DE)

(73) Assignee: Henkel AG & Co. KGaA, Duesseldorf (DE)

(21) Appl. No.: 16/718,080

(22) Filed: Dec. 17, 2019

(30) Foreign Application Priority Data
Dec. 18, 2018 (DE) 10 2018 222 047.6

(51) Int. Cl. A61K 8/73; A61Q 5/06; A61K 8/81

(52) U.S. Cl. CPC A61K 8/737; A61K 8/817; A61Q 5/06

(57) ABSTRACT

A cosmetic composition for temporarily styling keratinous fibres, containing:

a) at least one cationically modified guar derivative with a mass average molecular weight in the range from about 5000 to about 200000 and a cationic degree of substitution in the range from about 0.1 to about 2, and

c) at least one vinylcaprolactam-containing copolymer, its use and a method using a composition of this type.

### CROSS-REFERENCE TO RELATED APPLICATION

This application claims priority to German Patent Application No. 10 2018 222 047.6, filed Dec. 18, 2018, which is incorporated herein by reference in its entirety.

### TECHNICAL FIELD

The present disclosure relates to a cosmetic composition on the basis of two selected polymers for setting hair or for temporarily styling keratinous fibres, in particular human hair, as well as to a method using this composition, and to its use.

### BACKGROUND

The temporary shaping of hairstyles for a longer period of me of up to several days usually requires the use of setting substances. In this regard, hair treatment agents which serve to temporarily shape the hair play an important role. Appropriate compositions for temporary styling usually contain synthetic polymers and/or waxes as the setting substance. Compositions for supporting the temporary styling of keratinous fibres can, for example, be formulated as a hairspray, hair wax, hair gel or hair mousse.

The most important property of an agent for the temporary styling of hair, which will also be referred to below as a styling agent, is that the treated hair in its freshly modelled form—i.e. in which the hair is set—are provided with as strong a hold as possible. This is also known as a strong hold or a firm hold of the styling agent. The hold of the hairstyle is essentially determined by the type and quantity of the setting substance employed, although the other components of the styling agent may also have an influence.

In addition to a firm hold, styling agents must also comply with a whole series of other requirements. These can he broadly classified into properties on the hair, properties of the respective formulation, for example properties of the mousse, the gel or the sprayed aerosol, and properties which relate to handling of the styling agent; the properties on the hair are of particular importance. Moisture resistance, low tack and a well-balanced conditioning effect should be mentioned in particular. Furthermore, a styling agent should be able to be used for all types of hair and be gentle on the hair and skin.

The hold of a hairstyle in general, and with curled hair the curl retention, are particular challenges for styling agents. What is known as curl retention is a measure of the degree of hold of the curls. Normally, the curl retention is poorer when the treated hair is in moist surroundings, because the tendency of the hair to absorb moisture, i.e. water, deteriorates the retention of the curls.

in order to meet the various challenges, a large number of synthetic polymers for use in styling agents as setting substances have already been developed. The polymers can be categorized into cationic, anionic, non-ionic and amphoteric setting polymers.

A styling agent on the basis of a selected polymer combination comprising a polymer with the INCI name Vinyl Caprolactam/VP/Dimethylaminoethyl Methacrylate Copolymer has been described in the international patent application WO 2013/091993 A1.

### BRIEF SUMMARY

One aim of the present disclosure is to provide further suitable combinations of polymers which are exemplified by good film-forming and/or setting properties, have a high degree of hold without having to sacrifice flexibility and good moisture resistance—in particular perspiration and water resistance. A particular aim of the present disclosure it so provide styling agents of this type which provide both good long term hold and good humidity curl retention.

As contemplated herein, this has been achieved by employing a combination of two selected polymers.

The present disclosure provides the following:

A cosmetic composition for temporarily styling keratinous fibres, containing:

a) at least one cationically modified guar derivative with a mass average molecular

weight in the range from about 5000 to about 200000 and a cationic degree of substitution in the range from about 0.1 to about 2, and

b) at least one vinylcaprolactam-containing copolymer.

The cosmetic composition as contemplated herein, wherein the proportion by weight of the cationically modified guar derivative a) with respect to the total weight of the composition is from about 0.1% to about 10% by weight, preferably from about 0.15% to about 5% by weight and in particular from about 0.2% to about 2.5% by weight.

The cosmetic composition as contemplated herein, wherein the cationically modified guar derivative a) has a mass average molecular weight in the range from about 20000 to about 150000, more preferably in the range from about 35000 to about 100000 and more particularly preferably in the range from about 50000 to about 70000.

The cosmetic composition as contemplated herein, wherein the cationically modified guar derivative a) has a cationic degree of substitution in the range from about 0.2 to about 1.

The cosmetic composition as contemplated herein, wherein the cationically modified guar derivative a) is selected from the group of compounds with the INCI name Guar Hydroxypropyltrimonium Chloride.

The cosmetic composition as contemplated herein, wherein the proportion by weight of the copolymer b) with respect to the total weight of the composition is from about 0.1% to about 10% by weight, preferably from about 0.15% to about 5% by weight and in particular from about 0.2% to about 2.5% by weight.

The cosmetic composition as contemplated herein, wherein the copolymer b) is obtained by reacting N-vinylcaprolactam with N-vinylpyrrolidone and N,N-dimethylaminoethylmethacrylate.

The cosmetic composition as contemplated herein, wherein a terpolymer of N-vinylcaprolactam, N-vinylpyrrolidone and N,N-dimethylaminoethylmethacrylate is used as the copolymer b).

The cosmetic composition as contemplated herein, wherein the copolymer b) is selected from the group of compounds with the INCI name Vinyl Caprolactam/VP/Dimethylaminoethyl Methacrylate Copolymer.

The cosmetic composition as contemplated herein, wherein the copolymer b) is obtained by reacting N-vinylcaprolactam with N-vinylpyrrolidone and N,N-dimethylaminopropylmethacrylamide.

The cosmetic composition as contemplated herein, wherein a terpolymer of N-vinylcaprolactam, N-vinylpyrrolidone and N,N-dimethylaminopropylmethacrylamide is used as the copolymer b).

The cosmetic composition as contemplated herein, wherein the copolymer b) is selected from the group of compounds with the INCI name VP/Vinyl Caprolactam/DMAPA Acrylates Copolymer.

The cosmetic composition as contemplated herein, wherein the weight ratio of cationically modified guar derivative a) to copolymer b) is from about 10:1 to about 1:10, preferably from about 5:1 to about 1:5, more preferably from about 3:1 to about 1:3 and particularly preferably from about 1.1:1 to about 1:1.1.

The cosmetic composition as contemplated herein, wherein it further contains c) polyvinylpyrrolidone and/or vinylpyrrolidone/vinyl acetate copolymer, preferably polyvinylpyrrolidone.

The cosmetic composition as contemplated herein, wherein the proportion by weight of the polyvinylpyrrolidone and/or vinylpyrrolidone/vinyl acetate copolymer c) to the total weight of the cosmetic composition is from about 0.1% to about 10% by weight, preferably from about 2% to about 8.5% by weight and in particular from about 3% to about 7% by weight.

The cosmetic composition as contemplated herein, wherein with respect to its total weight, the composition contains from about 0.01% to about 5% by weight, preferably from about 0.02% to about 4% by weight and in particular from about 0.05% to about 2% by weight of an organic acid, preferably lactic acid.

The cosmetic composition as contemplated herein, wherein with respect to its total weight, the composition contains at least about 20% by weight, preferably at least about 40% by weight and in particular at least about 65% by weight of water.

The cosmetic composition as contemplated herein, wherein the composition is in the form of a hair gel, hair spray, hair foam, hair cream or hair wax.

Use of a cosmetic composition as contemplated herein, for temporarily styling keratin-containing fibres, in particular human hair.

Use of a cosmetic composition as contemplated herein, for improving the moisture resistance of temporarily styled keratinous fibres.

Use of a cosmetic composition as contemplated herein, for improving the high humidity curl retention of temporarily styled keratinous fibres.

A method for temporarily styling keratin-containing fibres, in particular human hair, in which a cosmetic composition as contemplated herein is applied to the keratinous fibres and they are temporarily set into their shape.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Cationic guar derivatives are used in hair care products in order to care for the hair, for example to endow the hair with improved combability. In skin care products, cationic guar derivatives can be used to provide the skin with conditioning effects. In detergent and fabric softener formulations, cationic guar derivatives provide the fabrics treated with them with conditioning, softening, wear-resistant and antistatic properties.

It has more surprisingly been shown that selected cationic guar derivatives in combination with a setting/film-forming styling polymer, the vinylcaprolactam-containing polymer b), which is already used in styling products, can produce a firm hold in the styling products. Other usually required properties of styling products such as low tack are retained thereby. A good combination of properties of this type was not expected, even with a knowledge of the individual components, and was surprising. It has been shown that by employing the combination of the two components, a strong supra-additive, i.e. synergistic effect, was obtained as regards the moisture resistance, in particular as regards the high humidity curl retention, which has been demonstrated by the HHCR test (high humidity curl retention test).

The term “keratinous fibres” as used as contemplated herein encompasses fur, wool and feathers, but in particular human hair. In this regard, “human hair” encompasses hair of the head and/or beard.

The essential components of the cosmetic composition are the cationic guar derivative a) and the vinylcaprolactarn-containing copolymer b).

The cosmetic compositions are distinguished from alternative cosmetic compositions by an improved high humidity curl retention in particular. A weight ratio for the cationically modified guar derivative a) and for the copolymer b) in the cosmetic composition of from about 10:1 to about 1:10, preferably of from about 5:1 to about 1:5 and in particular of from about 3:1 to about 1:3 has been shown to be particularly advantageous as regards the cosmetic properties of the compositions. Particularly advantageously, the weight ratio of the cationically modified guar derivative a) to the copolymer b) is about 1:1, in particular in the range from about 1.1:1 to about 1:1.1.

The cosmetic compositions contain a cationic guar derivative a) as the first mandatory component.

In the context of this application, the term “guar derivative” should be understood to mean (bio)chemically and/or physically modified guarans. Guaran is a polysaccharide formed from galactose and mannose and has a linear backbone of β-1,4-bonded mannose units. Alternate mannose units of this backbone are bonded to galactose units via β-1,6-glycoside bonds. The chemical modification of these guarans may, for example, be carried out by esterification or etherification of the hydroxy groups of the polysaccharide or by reaction with bases, acids or oxidizing agents. A biochemical modification of these guarans may, for example, be carried out by reaction with hydrolytic enzymes, bacteria or fungi. An example of a physical modification is the use of heat, radiation and comminution, for example using a high speed mixer.

The term “cationically modified guar derivatives” should be understood to mean guarans the hydroxy groups of which have been esterified or etherified with a compound which contains at least one cationic group. These cationic groups may be either permanently cationic or temporarily cationic. The term “permanently cationic” as used as contemplated herein indicates compounds which contain a cationic group independently of the pH of the cosmetic composition. This in particular includes compounds containing quaternary nitrogen atoms, such as quaternary ammonium groups. In contrast, “temporarily cationic” indicates those compounds which only have a cationic group at specific pHs, in particular pHs in the acid range. Examples of temporarily cationic groups are amine groups.

The cationic functionality can be introduced using a wide variety of methods. Thus, for example, the starting material may be reacted for a sufficient period and at a sufficient temperature with a tertiary amine compound or a quaternary ammonium compound which contains groups which are capable of reacting with reactive groups in the guar, in particular the hydroxy groups.

Examples of suitable compounds for introducing the cationic functionality include 2-dialkylaminoethylchloride and quaternary ammonium compounds such as 3-chloro-2-hydroxypropyl trimethylammonium chloride and 2,3-epoxypropyltrimethylammonium chloride. Other examples are glycidyltrialkylammonium salts and 3-halogen-2-hydroxypropyltrialkylammonium salts such as glycidyltrimethylammonium chloride, glycidyltriethylammonium chloride, glycidyltripropylammonium chloride, glycidylethyldimethylammonium chloride, glycidyldiethylmethylammonium chloride and the corresponding bromides and iodides; 3-chloro-2-hydroxypropyltrimethylammonium chloride, 3-chloro-2-hydroxypropyltriethylammonium chloride, 3 -chloro-2-hydroxypropyltripropylammonium chloride, 3 -chloro-2-hydroxypropylethyldimethylammonium chloride and their corresponding bromides and iodides; and quaternary ammonium compounds such as halides of compounds containing an imidazoline ring.

Preferably, the cationically modified guar derivative a) comprises at least one structural unit with formula (I):

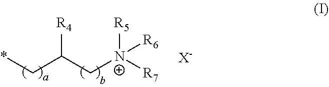

in which
R₄ represents hydrogen, a C₁₋₄ alkyl group or a hydroxy group,
R₅, R₆ and R₇ respectively independently of one another represent a C₁₋₈ alkyl group,
a and b respectively independently of one another represent whole numbers from 1 to 3,
X— represents a physiologically acceptable anion.

Examples of a C₁₋₄ alkyl group are methyl, ethyl, propyl, isopropyl, butyl, sec-butyl, isobutyl or tert-butyl groups.

Examples of a C₁₋₈ alkyl group are methyl, ethyl, propyl, isopropyl, butyl, sec-butyl, isobutyl, tert-butyl, pentyl and hexyl, heptyl and octyl groups.

Preferably, in the structural unit with formula (I), the residue R₄ represents a hydroxy group and a and b, respectively independently of one another, represent the integer 1.

Furthermore, preferably, in the structural unit with formula (I), the residues R₅ to R₇, respectively independently of one another, represent a C₁₋₆ alkyl group, preferably a C₁₋₄ alkyl group, preferably a C₁₋₃ alkyl group, in particular a C₁ alkyl group, and X^{—} represents a halide ion, in particular chloride.

Other derivatives of the cationic guar derivative with non-ionic substituents, i.e. hydroxyalkyl, wherein the alkyl represents a linear or branched carbohydrate residue containing 1 to 6 carbon atoms (for example hydroxyethyl, hydroxypropyl, hydroxybutyl), or anionic substituents such as carboxymethyl groups, are optional. These optional substituents may be added to the cationic guar derivatives by reaction with reagents such as (1) alkylene oxides (for example ethylene oxide, propylene oxide, butylene oxide) in order to obtain hydroxyethyl groups, hydroxypropyl groups or hydroxybutyl groups, or with (2) chloromethylacetic acid, in order to obtain a carboxymethyl group. Extremely preferably, however, the cationic guar derivative a) does not contain any further substituents, irrespective of whether they are non-ionic or anionic in nature.

The cationically modified guar derivative a) has a mass average molecular weight in the range from about 5000 to about 200000. Preferably, the cationically modified guar derivative a) has a mass average molecular weight in the range from about 20000 to about 150000, more preferably in the range from about 35000 to about 100000 and more particularly preferably in the range from about 50000 to about 70000.

The mass average molecular weight may, for example, be determined by employing gel permeation chromatography using a polystyrene standard.

The cationically modified guar derivative a) has a cationic degree of substitution (DS) in the range from about 0.1 to about 2. Preferably, the cationic degree of substitution is in the range from about 0.2 to about 1.

Particularly advantageously, the cationically modified guar derivative a) has a degree of substitution (DS) by the structural unit with formula (I) of from about 0.1 to about 2, in particular of from about 0.2 to about 1.

The degree of substitution (DS) describes the mean number of cationic structural units, in particular of cationic structural units with formula (I) which are bonded per monomer of polysaccharide, i.e. per anhydromannose and anhydrogalactose. Because each monomer of the polysaccharide contains an average of about 3 free OH groups, the DS value may take values between 0 and about 3. Thus, for example, a DS value of about 1 means that on average, one cationic structural unit per monomer is bonded to the polysaccharide, so that each monomer has a further about 2 free OH groups. The degree of substitution (DS) may, for example, be determined by employing ¹H-NMR spectroscopy or titration.

Extremely preferably, the cationically modified guar derivative comprises a cationically modified guar derivative with the INCI name “Guar Hydroxypropyltrimonium Chloride”, which has a mass average molecular weight in the range from about 5000 to about 200000 and a cationic degree of substitution in the range from about 0.1 to about 2. An example of a cationically modified guar derivative of this type is commercially available under the name “N-Hance CCG 45 Cationic Guar” ((INCI name: Guar Hydroxypropyltrimonium Chloride; about 92% by weight active substance in water) from Ashland Specialty Chemical.

The proportion by weight of the cationic guar derivative a) with respect to the total weight of the composition is preferably from about 0.1% to about 10% by weight, particularly preferably from about 0.15% to about 5% by weight and in particular from about 0.2% to about 2.5% by weight.

A second essential component of the cosmetic composition is the vinylcaprolactam-containing copolymer b).

The copolymers b) used in the cosmetic compositions are preferably non-ionic or temporarily cationic.

Preferred cosmetic compositions contain a copolymer b) with at least one structural unit with formula (M1) and at least one structural unit with formula (M2) and at least one structural unit with formula (M3):

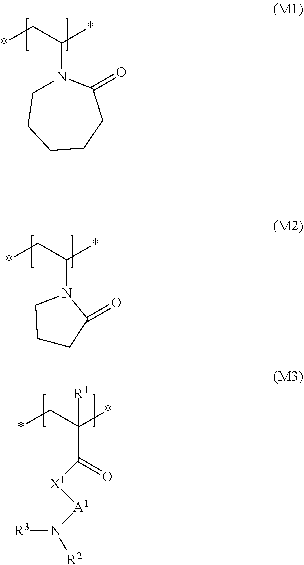

with
R¹ representing a hydrogen atom or a methyl group,
X¹ representing an oxygen atom or a NH group,
A¹ representing an ethan-1,2-diyl, propan-1,3-diyl or butan-1,4-diyl group,
R² and R³ , independently of each other, representing a (C₁ to C₄) alkyl group.
R¹ in formula (M3) preferably represents a methyl group.

Examples of (C₁ to C₄) alkyl groups in accordance with formula (M3) are methyl, ethyl, isopropyl, n-propyl, n-butyl, sec-butyl, isobutyl and tert-butyl. R² and R³ in formula (M3) preferably represent a methyl group.

More particularly suitable polymers in this embodiment have at least one structural unit with formula (M3), in which A¹ represents an ethan-1,2-diyl group and X¹ represents an oxygen atom.

More preferably, a polymer of this type preferably comprises at least one structural unit with formula (M1) and at least one structural unit with formula (M2) and at least one structural unit with formula (M3-1):

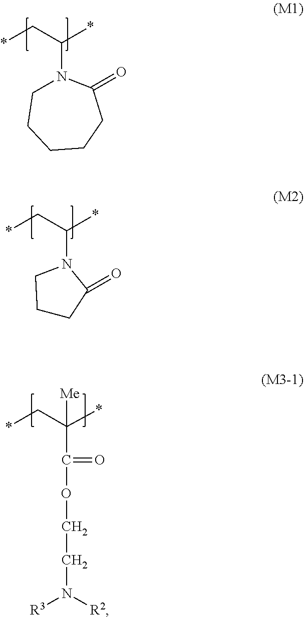

in which R² and R³, independently of each other, represent a (C₁ to C₄) alkyl group, in particular methyl. Examples of (C₁ to C₄) alkyl groups in accordance with formula (M3-1) are methyl, ethyl, isopropyl, n-propyl, n-butyl, sec-butyl, isobutyl and tert-butyl.

More particularly preferably, the cosmetic composition contains a terpolymer formed from N-vinylcaprolactam, N-vinylpyrrolidone and N,N-dimethylaminoethylmethacrylate as copolymer b). Copolymers of this type may, for example, be obtained under the trade names Advantage LC-E (INCI name: Vinylcaprolactam/VP/Dimethylaminoethylmethacrylate Copolymer (and) Lauryl Pyrrolidone; about 37% by weight active substance in ethanol with the addition of N-lauryl pyrrolidone), Advantage S (INCI name: Vinylcaprolactam/VP/Dimethylaminoethylmethacrylate Copolymer, 100% by weight active substance, powder) or Advantage LC-A (INCI name: Vinylcaprolactam/VP/Dimethylaminoethylmethacrylate Copolymer; about 37% by weight active substance in ethanol) from Ashland.

In a second preferred embodiment, the copolymer b) is obtained by copolymerization of the monomers N-vinylpyrrolidone, N-vinylcaprolactam as well as N,N-dimethylaminopropylmethacrylamide.

Preferred copolymers b) include at least about 90% by weight, preferably at least about 95% by weight and in particular at least about 97% by weight of the monomers N-vinylpyrrolidone, N-vinylcaprolactam and N,N-dimethylaminopropylmethacrylamide. Particularly preferred copolymers b) were obtained exclusively from the monomers N-vinylpyrrolidone, N-vinylcaprolactam and N,N-dimethylaminopropylmethacrylamide. Examples of copolymers of this type are available under the trade names Aquaflex SF 40 (INCI name: VP/Vinyl Caprolactam/DMAPA Acrylates Copolymer, Alcohol Denat.; 38-42% by weight active substance in ethanol) from ISP.

The proportion by weight of the copolymer b) with respect to the total weight of the composition is preferably from about 0.1% to about 10% by weight, particularly preferably from about 0.15% to about 5% by weight and in particular from about 0.2% to about 2.5% by weight.

It may be preferable for the cosmetic composition to contain one more or several more other polymer(s) which is/are different from the polymers a) and b) and, for example, promote thickening agent or gel formation or film formation. Examples are cationic, anionic, non-ionic or amphoteric polymers.

Examples are Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-hydroxypropyl Dimethicone Acrylate Copolymer, Isopropyl Ester of PVM/MA Copolymer, Lauryl Acrylate Crosspolymer, Lauryl methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polyquaternium-68, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium isophthalic Acid Sulfonate/Glycol Copolymer, Trimethylolpropane Triacrylate, Trimethylsiloxysilylcarbamoyl Pullulan, VA/Crotonates Copolymer, VA/Crotonates/methacryloxybenzophenone-1 Copolymer, VA/Crotonates/Vinyl Neodecanoate Copolymer, VA/Crotonates/Vinyl propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, Yeast Palmitate and Styrene/VP Copolymer.

Preferably, the further component which acts as a gel-forming agent is a homopolyacrylic acid (INCI: Carbomer), which is commercially available in a variety of embodiments, for example under the name Carbopol®. The carbomer is preferably present in a proportion of from about 0.02% to about 3% by weight, preferably from about 0.05% to about 1.5% by weight and more preferably from about 0.2% to about 0.8% by weight, with respect to the total weight of the cosmetic composition.

In order to further enhance their cosmetic action, in addition to the polymers a) and b) and an optionally added thickening agent or gel-forming agent, preferred compositions contain a film-forming polymer c) which differs from these ingredients, in particular an anionic or non-ionic polymer c).

Examples of non-ionic polymers are:
vinylpyrrolidone/vinyl ester-copolymers such as, for example, those marketed under the trademark Luviskol (BASF). Luviskol VA 64 and Luviskol VA 73, respectively vinylpyrrolidone/vinyl acetate copolymers, are preferred non-ionic polymers.
cellulose ethers such as hydroxypropyl cellulose, hydroxyethyl cellulose and methylhydroxypropyl cellulose, such as those marketed under the trademarks Culminal and Benecel (AQUALON).
shellac.
polyvinylpyrrolidones, such as those marketed under the trademark Luviskol (BASE).
siloxanes. These siloxanes may be both water-soluble as well as water-insoluble. Volatile as well as non-volatile siloxanes are suitable; the term “non-volatile siloxanes” should be understood to mean those compounds with a boiling point under normal pressure of over about 200° C. Preferred siloxanes are polydialkylsiloxanes such as, for example polydimethylsiloxane, polyalkylaryl siloxanes such as polyphenylmethyl siloxane, for example, ethoxylated polydialkyl siloxanes as well as polydialkyl siloxanes which contain amine and/or hydroxy groups.
glycosidic substituted silicones.

Because of its cosmetic action in combination with the copolymers (a) and (b), as contemplated herein, preferred film-forming polymers are polyvinylpyrrolidones (INCI name: PVP) as well as vinylpyrrolidone/vinyl acetate copolymers (INCI name VP/VA Copolymer). By adding film-forming polymers, in particular the aforementioned polyvinylpyrrolidones and vinylpyrrolidone/vinyl acetate copolymers, primarily the holding properties but also the application properties for the cosmetic compositions are advantageously influenced. The proportion by weight of these polymers is preferably limited to quantities of between about 1.0% and about 10% by weight. Preferred cosmetic compositions contain, with respect to their total weight, a further from about 1% to about 10% by weight of polyvinylpyrrolidone and/or vinylpyrrolidone/vinyl acetate copolymer, preferably polyvinylpyrrolidone. Particularly preferred cosmetic agents have a proportion by weight of polyvinylpyrrolidone and/or vinylpyrrolidone/vinyl acetate copolymer c) with respect to the total weight of the cosmetic composition of from about 2% to about 8.5% by weight, preferably from about 3% to about 7.0% by weight.

The cosmetic composition as contemplated herein may contain further normal ingredients for styling products. Additional care products may in particular be mentioned as the further suitable auxiliary products and additives.

The composition may, for example, contain at least one protein hydrolysate and/or a derivative thereof as the care product. Protein hydrolysates are product mixtures which are obtained by the acidic, basic or enzymatically catalysed decomposition of proteins. The term “protein hydrolysate” as used in the present disclosure should also be understood to include total hydrolysates as well as individual amino acids and their derivatives, as well as mixtures of the various amino acids.

The composition may also contain at least one vitamin, a provitamin, a vitamin precursor and/or one of its derivatives as the care product. In this regard, as contemplated herein, those vitamins, provitamins and vitamin precursors are preferred which are usually categorized into the groups A, B, C, E, F and H.

Like the addition of glycerine and/or propylene glycol, the addition of panthenol increases the flexibility of the polymer film formed when using the composition.

The compositions may also contain at least one plant extract as the care product, as well as mono- or oligosaccharides and/or lipids.

Furthermore, oily substances are suitable as the care product. Examples of natural and synthetic cosmetic oily substances include vegetable oils, liquid paraffin oils, isoparaffin oils and synthetic hydrocarbon oils as well as di-n-alkyl ethers containing a total of 12 to 36 carbon atoms, in particular 12 to 24 carbon atoms. Preferred cosmetic compositions contain at least one oily substance, preferably at least one oily substance from the group formed by silicone oils. Particular examples of the silicone oil group include dimethicones, which also includes cyclomethicones, amino-functionalized silicones as well as dimethicone oils. The dimethicones may be both linear and branched, as well as cyclic or cyclic and branched. Particularly suitable silicone oils or silicone gums are dialkyl and alkylaryl siloxanes such as, for example, dimethylpolysiloxane and methylphenylpolysiloxane, as well as their alkoxylated, quaternized and also anionic derivatives, Cyclic and linear polydialkylsiloxanes are preferred, as well as their alkoxylated and/or aminated derivatives, dihydroxypolydimethy siloxanes and polyphenylalkylsiloxanes.

Ester oils, i.e. esters of C₆-C₃₀ fatty acids with C₂-C₃₀ fatty alcohols, preferably monoesters of fatty acids with alcohols containing 2 to 24 C atoms such as, for example, isopropyhnyristate (Rilanit® IPM), isononanoic acid-C16-18-alkylester (Cetiol® SN), 2-ethylhexyl palmitate (Cegesoft® 24), stearic acid-2-ethylhexylester (Cetiol® 868), cetyl oleate, glycerine tricaprylate, coconut oil alcohol caprinatelcaprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V), are further preferred oily care substances.

Dicarboxylic acid esters, symmetrical, asymmetrical or cyclic esters of carboxylic acids with fatty alcohols, trifatty acid esters of saturated and/or unsaturated linear and/or branched fatty acids with glycerine or fatty acid partial glycerides, which should be understood to include monoglycerides, diglycerides and their technical mixtures, are also suitable as care substances.

Emulsifying agents or surfactants are also preferably contained in the composition. PEG derivatives of hydrogenated castor oil are preferred, for example those available under the names PEG Hydrogenated Castor Oil, for example PEG-30 Hydrogenated Castor Oil, PEG-33 Hydrogenated Castor Oil, PEG-35 Hydrogenated Castor Oil, PEG-36 Hydrogenated Castor Oil or PEG-40 Hydrogenated Castor Oil. The use of PEG-40 Hydrogenated Castor Oil is preferred in the present disclosure. These are preferably employed in a quantity of from about 0.05% to about 1.5% by weight, preferably from about 0.1% to about 1.0% by weight, more preferably from about 0.2% to about 0.8% by weight or from about 0.3% to about 0.6 by weight. By using surface-active substances, in particular the aforementioned PEG derivatives of hydrogenated castor oil, the rinsing properties of the cosmetic compositions are improved in addition to the formulation capabilities.

The cosmetic agents as contemplated herein contain the ingredients or substances in a cosmetically acceptable support.

Preferred cosmetically acceptable supports are aqueous, alcoholic or hydroalcoholic media which preferably contain at least about 10% by weight of water with respect to the total weight of the agent. Particularly preferably, the cosmetic support contains water, in particular in a quantity such that the cosmetic composition, with respect to its total weight, contains at least about 20% by weight, in particular at least about 40% by weight, and most preferably at least about 65% by weight of water. More particularly preferred cosmetic compositions exhibit a water content of from about 50% to about 95% by weight, preferably from about 60% to about 90% by weight and in particular from about 65% to about 85% by weight with respect to their total weight.

Particularly for cosmetic purposes, the alcohols employed may be the usually employed lower alcohols containing 1 to 4 carbon atoms such as ethanol and isopropanol, for example.

Examples of water-soluble solvents as co-solvents are glycerine and/or ethylene glycol and/or 1,2-propylene glycol in a quantity of from 0 to about 30% by weight with respect to the composition as a whole.

Preferably, the composition may contain an organic acid or its salt. The organic acid is preferably selected from the group including maleic acid, lactic acid, acetic acid, propanoic acid, citric acid, tartaric acid, succinic acid, oxalic acid, gluconic acid, malic acid, amino acids and mixtures thereof. More particularly preferably, the organic acid comprises lactic acid.

The quantity of organic acid, preferably of lactic acid, to the total weight of the cosmetic composition is preferably from about 0.01% to about 5% by weight, more preferably from about 0.02% to about 4% by weight and particularly preferably from about 0.05% to about 2% by weight.

The cationically modified guar derivative is preferably used in the form of an acidic aqueous solution. In order to acidify the aqueous solution, an organic acid is preferably employed.

The composition of some preferred cosmetic compositions may be discerned from the following tables (given as a % by weight with respect to the total weight of the cosmetic agent, unless stated otherwise).

| | Formula | Formula | Formula | Formula | Formula |
|---|---|---|---|---|---|
| | 1 | 2 | 3 | 4 | 5 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Misc | ad 100 | ad 100 | ad 100 | ad 100 | ad 100 |

| | Formula | Formula | Formula | Formula | Formula |
|---|---|---|---|---|---|
| | 1a | 2a | 3a | 4a | 5a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethylaminoethyl | | | | | |
| Methacrylate | | | | | |
| Copolymer | | | | | |
| Misc | ad 100 | ad 100 | ad 100 | ad 100 | ad 100 |

| | Formula | Formula | Formula | Formula | Formula |
|---|---|---|---|---|---|
| | 1b | 2b | 3b | 4b | 5b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| Misc | ad 100 | ad 100 | ad 100 | ad 100 | ad 100 |

| | Formula | Formula | Formula | Formula | Formula |
|---|---|---|---|---|---|
| | 1c | 2c | 3c | 4c | 5c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| Misc | ad 100 | ad 100 | ad 100 | ad 100 | ad 100 |

| | Formula | Formula | Formula | Formula | Formula |
|---|---|---|---|---|---|
| | 1d | 2d | 3d | 4d | 5d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| Misc | ad 100 | ad 100 | ad 100 | ad 100 | ad 100 |

| | Formula | Formula | Formula | Formula | Formula |
|---|---|---|---|---|---|
| | 6 | 7 | 8 | 9 | 10 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Polyvinylpyr- | 1 to 10 | 2 to 8.5 | 2 to 8.5 | 3 to 7 | 3 to 7 |
| rolidone | | | | | |
| Misc | ad 100 | ad 100 | ad 100 | ad 100 | ad 100 |

| | Formula | Formula | Formula | Formula | Formula |
|---|---|---|---|---|---|
| | 6a | 7a | 8a | 9a | 10a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethylaminoethyl | | | | | |
| Methacrylate | | | | | |
| Copolymer | | | | | |
| Polyvinylpyr- | 1 to 10 | 2 to 8.5 | 2 to 8.5 | 3 to 7 | 3 to 7 |
| rolidone | | | | | |
| Misc | ad 100 | ad 100 | ad 100 | ad 100 | ad 100 |

| | Formula | Formula | Formula | Formula | Formula |
|---|---|---|---|---|---|
| | 6b | 7b | 8b | 9b | 10b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| Polyvinylpyr- | 1 to 10 | 2 to 8.5 | 2 to 8.5 | 3 to 7 | 3 to 7 |
| rolidone | | | | | |
| Misc | ad 100 | ad 100 | ad 100 | ad 100 | ad 100 |

| | Formula | Formula | Formula | Formula | Formula |
|---|---|---|---|---|---|
| | 6c | 7c | 8c | 9c | 10c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| Polyvinylpyr- | 1 to 10 | 2 to 8.5 | 2 to 8.5 | 3 to 7 | 3 to 7 |
| rolidone | | | | | |
| Misc | ad 100 | ad 100 | ad 100 | ad 100 | ad 100 |

| | Formula | Formula | Formula | Formula | Formula |
|---|---|---|---|---|---|
| | 6d | 7d | 8d | 9d | 10d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| Polyvinylpyr- | 1 to 10 | 2 to 8.5 | 2 to 8.5 | 3 to 7 | 3 to 7 |
| rolidone | | | | | |
| Misc | ad 100 | ad 100 | ad 100 | ad 100 | ad 100 |

| | Formula | Formula | Formula | Formula | Formula |
|---|---|---|---|---|---|
| | 11 | 12 | 13 | 14 | 15 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| Misc | ad 100 | ad 100 | ad 100 | ad 100 | ad 100 |

| | Formula | Formula | Formula | Formula | Formula |
|---|---|---|---|---|---|
| | 11a | 12a | 13a | 14a | 15a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethylaminoethyl | | | | | |
| Methacrylate | | | | | |
| Copolymer | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| Misc | ad 100 | ad 100 | ad 100 | ad 100 | ad 100 |

| | Formula | Formula | Formula | Formula | Formula |
|---|---|---|---|---|---|
| | 11b | 12b | 13b | 14b | 15b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| Misc | ad 100 | ad 100 | ad 100 | ad 100 | ad 100 |

| | Formula | Formula | Formula | Formula | Formula |
|---|---|---|---|---|---|
| | 11c | 12c | 13c | 14c | 15c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| Misc | ad 100 | ad 100 | ad 100 | ad 100 | ad 100 |

| | Formula | Formula | Formula | Formula | Formula |
|---|---|---|---|---|---|
| | 11d | 12d | 13d | 14d | 15d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| Misc | ad 100 | ad 100 | ad 100 | ad 100 | ad 100 |

| | Formula | Formula | Formula | Formula | Formula |
|---|---|---|---|---|---|
| | 16 | 17 | 18 | 19 | 20 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Misc | ad 100 | ad 100 | ad 100 | ad 100 | ad 100 |

| | Formula | Formula | Formula | Formula | Formula |
|---|---|---|---|---|---|
| | 16a | 17a | 18a | 19a | 20a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethylaminoethyl | | | | | |
| Methacrylate | | | | | |
| Copolymer | | | | | |
| Carbomer | 0.02 to 3 | 0.05 to 2 | 0.05 to 1.5 | 0.2 to 1.5 | 0.2 to 0.8 |
| Misc | ad 100 | ad 100 | ad 100 | ad 100 | ad 100 |

| | Formula | Formula | Formula | Formula | Formula |
|---|---|---|---|---|---|
| | 16b | 17b | 18b | 19b | 20b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| Carbomer | 0.02 to 3 | 0.05 to 2 | 0.05 to 1.5 | 0.2 to 1.5 | 0.2 to 0.8 |
| Misc | ad 100 | ad 100 | ad 100 | ad 100 | ad 100 |

| | Formula | Formula | Formula | Formula | Formula |
|---|---|---|---|---|---|
| | 16c | 17c | 18c | 19c | 20c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| Carbomer | 0.02 to 3 | 0.05 to 2 | 0.05 to 1.5 | 0.2 to 1.5 | 0.2 to 0.8 |
| Misc | ad 100 | ad 100 | ad 100 | ad 100 | ad 100 |

| | Formula | Formula | Formula | Formula | Formula |
|---|---|---|---|---|---|
| | 16d | 17d | 18d | 19d | 20d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| Carbomer | 0.02 to 3 | 0.05 to 2 | 0.05 to 1.5 | 0.2 to 1.5 | 0.2 to 0.8 |
| Misc | ad 100 | ad 100 | ad 100 | ad 100 | ad 100 |

| | Formula | Formula | Formula | Formula | Formula |
|---|---|---|---|---|---|
| | 21 | 22 | 23 | 24 | 25 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Misc | ad 100 | ad 100 | ad 100 | ad 100 | ad 100 |

| | Formula | Formula | Formula | Formula | Formula |
|---|---|---|---|---|---|
| | 21a | 22a | 23a | 24a | 25a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethylaminoethyl | | | | | |
| Methacrylate | | | | | |
| Copolymer | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Misc | ad 100 | ad 100 | ad 100 | ad 100 | ad 100 |

| | Formula | Formula | Formula | Formula | Formula |
|---|---|---|---|---|---|
| | 21b | 22b | 23b | 24b | 25b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Misc | ad 100 | ad 100 | ad 100 | ad 100 | ad 100 |

| | Formula | Formula | Formula | Formula | Formula |
|---|---|---|---|---|---|
| | 21c | 22c | 23c | 24c | 25c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Misc | ad 100 | ad 100 | ad 100 | ad 100 | ad 100 |

| | Formula | Formula | Formula | Formula | Formula |
|---|---|---|---|---|---|
| | 21d | 22d | 23d | 24d | 25d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Misc | ad 100 | ad 100 | ad 100 | ad 100 | ad 100 |

| | Formula | Formula | Formula | Formula | Formula |
|---|---|---|---|---|---|
| | 26 | 27 | 28 | 29 | 30 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 26a | 27a | 28a | 29a | 30a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethylaminoethyl | | | | | |
| Methacrylate | | | | | |
| Copolymer | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 26b | 27b | 28b | 29b | 30b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 26c | 27c | 28c | 29c | 30c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 26d | 27d | 28d | 29d | 30d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 31 | 32 | 33 | 34 | 35 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Polyvinylpyr- | 1 to 10 | 2 to 8.5 | 2 to 8.5 | 3 to 7 | 3 to 7 |
| rolidone | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 31a | 32a | 33a | 34a | 35a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethylaminoethyl | | | | | |
| Methacrylate | | | | | |
| Copolymer | | | | | |
| Polyvinylpyr- | 1 to 10 | 2 to 8.5 | 2 to 8.5 | 3 to 7 | 3 to 7 |
| rolidone | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 31b | 32b | 33b | 34b | 35b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| Polyvinylpyr- | 1 to 10 | 2 to 8.5 | 2 to 8.5 | 3 to 7 | 3 to 7 |
| rolidone | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 31c | 32c | 33c | 34c | 35c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| Polyvinylpyr- | 1 to 10 | 2 to 8.5 | 2 to 8.5 | 3 to 7 | 3 to 7 |
| rolidone | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 31d | 32d | 33d | 34d | 35d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| Polyvinylpyr- | 1 to 10 | 2 to 8.5 | 2 to 8.5 | 3 to 7 | 3 to 7 |
| rolidone | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 36 | 37 | 38 | 39 | 40 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 36a | 37a | 38a | 39a | 40a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethylaminoethyl | | | | | |
| Methacrylate | | | | | |
| Copolymer | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 36b | 37b | 38b | 39b | 40b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 36c | 37c | 38c | 39c | 40c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 36d | 37d | 38d | 39d | 40d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 41 | 42 | 43 | 44 | 45 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 41a | 42a | 43a | 44a | 45a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethylaminoethyl | | | | | |
| Methacrylate | | | | | |
| Copolymer | | | | | |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 41b | 42b | 43b | 44b | 45b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 41c | 42c | 43c | 44c | 45c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 41d | 42d | 43d | 44d | 45d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| Carbomer | 0.02 to 3 | 0.05 to 2 | 0.05 to 1.5 | 0.2 to 1.5 | 0.2 to 0.8 |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 46 | 47 | 48 | 49 | 50 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 46a | 47a | 48a | 49a | 50a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethylaminoethyl | | | | | |
| Methacrylate | | | | | |
| Copolymer | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 46b | 47b | 48b | 49b | 50b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 46c | 47c | 48c | 49c | 50c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 46d | 47d | 48d | 49d | 50d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 51 | 52 | 53 | 54 | 55 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Polyvinylpyr- | 1 to 10 | 2 to 8.5 | 2 to 8.5 | 3 to 7 | 3 to 7 |
| rolidone | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 51a | 52a | 53a | 54a | 55a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethylaminoethyl | | | | | |
| Methacrylate | | | | | |
| Copolymer | | | | | |
| Polyvinylpyr- | 1 to 10 | 2 to 8.5 | 2 to 8.5 | 3 to 7 | 3 to 7 |
| rolidone | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 51b | 52b | 53b | 54b | 55b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| Polyvinylpyr- | 1 to 10 | 2 to 8.5 | 2 to 8.5 | 3 to 7 | 3 to 7 |
| rolidone | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |
| | Formula | Formula | Formula | Formula | Formula |
| | 51c | 52c | 53c | 54c | 55c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| Polyvinylpyr- | 1 to 10 | 2 to 8.5 | 2 to 8.5 | 3 to 7 | 3 to 7 |
| rolidone | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 51d | 52d | 53d | 54d | 55d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| Polyvinyl- | 1 to 10 | 2 to 8.5 | 2 to 8.5 | 3 to 7 | 3 to 7 |
| pyrrolidone | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 56 | 57 | 58 | 59 | 60 |
| Polymer a)* | 0.1 to 10 | 0.15 to 5 | 0.15 to 5 | 0.2 to 2.5 | 0.2 to 2.5 |
| Copolymer b) | 0.1 to 10 | 0.15 to 5 | 0.15 to 5 | 0.2 to 2.5 | 0.2 to 2.5 |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 56a | 57a | 58a | 59a | 60a |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| Vinyl | 0.1 to 10 | 0.15 to 5 | 0.15 to 5 | 0.2 to 2.5 | 0.2 to 2.5 |
| Caprolactam/VP/ | | | | | |
| Dimethyaminoethyl | | | | | |
| Methacrylate | | | | | |
| Copolymer | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 56b | 57b | 58b | 59b | 60b |
| N-Hance CCG 45 | 0.1 to 10 | 0.15 to 5 | 0.15 to 5 | 0.2 to 2.5 | 0.2 to 2.5 |
| (as solids content) | | | | | |
| Advantage ® S | 0.1 to 10 | 0.15 to 5 | 0.15 to 5 | 0.2 to 2.5 | 0.2 to 2.5 |
| (as solids content) | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 56c | 57c | 58c | 59c | 60c |
| Guar Hydroxy- | 0.1 to 10 | 0.15 to 5 | 0.15 to 5 | 0.2 to 2.5 | 0.2 to 2.5 |
| propyltrimonium | | | | | |
| Chloride** | | | | | |
| VP/Vinyl | 0.1 to 10 | 0.15 to 5 | 0.15 to 5 | 0.2 to 2.5 | 0.2 to 2.5 |
| Caprolactam/ | | | | | |
| DMAPA Acrylates | | | | | |
| Copolymer | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

| | Formula | Formula | Formula | Formula | Formula |
|---|---|---|---|---|---|
| | 56d | 57d | 58d | 59d | 60d |
| N-Hance CCG 45 | 0.1 to 10 | 0.15 to 5 | 0.15 to 5 | 0.2 to 2.5 | 0.2 to 2.5 |
| (as solids content) | | | | | |
| Aquaflex ® SF 40 | 0.1 to 10 | 0.15 to 5 | 0.15 to 5 | 0.2 to 2.5 | 0.2 to 2.5 |
| (as solids content) | | | | | |
| Vinylpyrrolidone/ | 1 to 10 | 2 to 8.5 | 2 to 8.5 | 3 to 7 | 3 to 7 |
| Vinyl acetate | | | | | |
| Copolymer | | | | | |
| PEG-40 | 0.05 to 1.5 | 0.1 to 1 | 0.2 to 0.8 | 0.3 to 0.8 | 0.3 to 0.6 |
| Hydrogenated | | | | | |
| Castor Oil | | | | | |
| Water | 50 to 95 | 50 to 95 | 60 to 90 | 60 to 90 | 65 to 85 |
| Misc | ad 100 | ad 100 | ad 100 | ad 100 | ad 100 |

* cationically modified guar derivative with a mass average molecular weight of from about 5000 to about 200000 and a cationic degree of substitution (DS) of from about 0.1 to about 2

** with a mass average molecular weight of from about 5000 to about 200000 and a cationic degree of substitution (DS) of from about 0.1 to about 2

The term “mise” should be understood to mean a cosmetic support, in particular (unless separately mentioned) water and, if appropriate, other usual components of styling products.

The cosmetic composition of the present disclosure may be formulated in the usual forms for temporary hair styling products, for example as a hair gel, hairspray, hair mousse or hair wax, Formulation as a hair gel is preferred,

Both hair mousses and hairsprays require the presence of propellants. As contemplated herein, however, preferably, no or only small quantities of hydrocarbons should be used in this regard. Propane, propane/butane mixtures and dimethyl ether are particularly suitable propellants for use in the present disclosure.

The present disclosure also concerns the use of cosmetic compositions as contemplated herein for the temporary styling of keratinous fibres, in particular human hair, as well as a method for temporarily styling keratinous fibres, in particular human hair, in which the cosmetic composition as contemplated herein is applied to the keratinous fibres and they are temporarily set into their shape.

The statements provided regarding the cosmetic compositions are also applicable mutatis mutandis to the further embodiments of the use and the method.

In a further aspect, this patent application concerns the use of a cosmetic composition as contemplated herein to improve the high humidity curl retention (HHCR) of temporarily styled keratinous fibres.

### EXAMPLES

II. The following hair gels were produced:

| Component/raw | INCI name or | | | |
|---|---|---|---|---|
| material | chemical name | V1 | V2 | E1 |
| N-Hance CCG 45 ¹ | Guar Hydroxy- | 1 | — | 0.5 |
| | propyltrimonium | | | |
| | Chloride | | | |
| Advantage ® S ² | Vinyl | — | 1 | 0.5 |
| | Caprolactam/VP/ | | | |
| | Dimethylaminoethyl | | | |
| | Methacrylate | | | |
| | Copolymer | | | |
| Water | | 99 | 99 | 99 |
| Total | | 100 | 100 | 100 |

¹ 92% by weight of active substance in water
² 100% by weight of active substance in water

The quantities in the table are given as a by % weight of the respective raw material, with respect to the total composition.

For the styling agents obtained, the high humidity curl retention was determined for clean Kerling hair strands using a HHCR test (high humidity curl retention test: 6 h) (mean value in each case determined for 5 hair strands):

| | V1 | V2 | E1 |
|---|---|---|---|
| HHCR | 19.4% | 53.6% | 60.3% |

The polymer combination E1 as contemplated herein thus exhibited a substantial supra-additive, synergistic effect as regards the high humidity curl retention.

II. The following further hair gels were produced:

| Component/raw | INCI name or | | | |
|---|---|---|---|---|
| material | chemical name | V1 | V2 | E1 |
| N-Hance CCG 45 ¹ | Guar Hydroxy- | 1 | — | 0.5 |
| | propyltrimonium | | | |
| | Chloride | | | |
| Aquaflex ® SF 40 ² | VP/Vinyl | — | 2.5 | 1.25 |
| | Caprolactam/ | | | |
| | DMAPA Acrylates | | | |
| | Copolymer | | | |
| Water | | 99 | 97.5 | 98.25 |
| Total | | 100 | 100 | 100 |

¹ 92% by weight of active substance in water
² 40% by weight of active substance in water

The quantities in the table are given as a % by weight of the respective raw material, with respect to the total composition.

For the styling agents obtained, the high humidity curl retention was determined for clean Kerling hair strands using a HHCR test (high humidity curl retention test: 6 h) (mean value in each case determined for 5 hair strands):

| | V1 | V2 | E1 |
|---|---|---|---|
| HHCR | 19.4% | 38.9% | 46.9% |

The polymer combination E1 as contemplated herein thus exhibited a substantial supra-additive, synergistic effect as regards the high humidity curl retention.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for temporarily styling keratinous fibres, comprising:
a) at least one cationically modified guar derivative with a mass average molecular weight in the range from about 5000 to about 200000 and a cationic degree of substitution in the range from 0.1 to 2, and
b) at least one vinylcaprolactam-containing copolymer.

2. The cosmetic composition as claimed in claim 1, wherein the proportion by weight of the cationically modified guar derivative a) with respect to the total weight of the composition is from about 0.1% to about 10% by weight.

3. The cosmetic composition as claimed in claim 1, wherein the cationically modified guar derivative a) is selected from the group of compounds with the INCI name Guar Hydroxypropyltrimonium Chloride.

4. The cosmetic composition as claimed in claim 1, wherein the proportion by weight of the copolymer b) with respect to the total weight of the composition is from about 0.1% to about 12% by weight.

5. The cosmetic composition as claimed in claim 1, wherein the copolymer b) is selected from the group of compounds with the INCI name Vinyl Caprolactam/VP/Dimethylaminoethyl Methacrylate Copolymer.

6. The cosmetic composition as claimed in claim 1, wherein the copolymer b) is selected from the group of compounds with the INCI name VP/Vinyl Caprolactam/DMAPA Acrylates Copolymer.

7. The cosmetic composition as claimed in claim 1, wherein the composition further comprises an organic acid.

8. The cosmetic composition as claimed in claim 1, wherein with respect to its total weight, the composition comprises at least about 20% by weight of water.

9. Use of a cosmetic composition as claimed in claim 1 for temporarily styling keratin-containing fibres.

10. A method for temporarily styling keratin-containing fibres, in which a cosmetic composition as claimed in claim 1 is applied to the keratinous fibres and they are temporarily set into their shape.
